(19) United States
(12) Patent Application Publication
(10) Pub. No.: US 2026/0311824 A1
(43) Pub. Date: Oct. 8, 2026

(54) SYSTEM FOR SURVEYING THE HEALTH AND/OR PERFORMANCE STATE OF A SUBJECT

(71) Applicant: Math Biology S.R.L., Roma (IT)

(72) Inventors: Raffaele MACCIONI, Roma (IT); Giuseppe SGRO, Roma (IT)

(73) Assignee: Math Biology S.R.L., Roma (IT)

(21) Appl. No.: 19/481,660

(22) PCT Filed: May 2, 2024
(86) PCT No.: PCT/EP2024/062130

§ 371 Date: Nov. 5, 2025

(30) Foreign Application Priority Data
May 5, 2023 (IT) 102023000008940

(51) Int. Cl. G16H 10/20; G16H 50/20; G16H 50/30

(52) U.S. Cl. CPC G16H 10/20; G16H 50/20; G16H 50/30

(57) ABSTRACT

A system (100) for submitting a subject to a survey aimed at assessing a health state and/or a performance state of the subject. The system comprises: one or more biosensors (105) adapted to detect, in one or more points of the body surface, a plurality of bioelectric signals emitted by the body of the subject and associated with at least one body zone dependent on a purpose of the survey; an acquisition module (120) configured to acquire said plurality of bioelectric signals; one or more substances (S) to which the subject has to be exposed during the execution of the survey, wherein the one or more substances are configured for inducing, by exposure of the subject thereto, reactions in one or more molecules that are involved in one or more biochemical reactions that constitute a metabolic chain in the at least one body zone; an analysis apparatus (140). The analysis apparatus is configured to implement one or more mathematical algorithms to: determine a metabolic condition of the subject according to a difference between first bioelectric signals, among said plurality of bioelectric signals, spontaneously emitted by the body of the subject in the absence of exposure of the subject to said one or more substances, and second bioelectric signals, among said plurality of bioelectric signals, emitted by the body of the subject in response to the exposure of the subject to said one or more substances, wherein the exposure of the subject to said one or more substances comprises the placement of the one or more substances externally to the body of the subject and in proximity or contact with the body of the subject; identify a correlation between the metabolic condition of the subject and at least one among nutritional data and/or sport medicine data, statistical data associated with one or more pathologies, diagnostic/clinical data of the subject obtained through one or more medical devices (130), measurements of vital signs of the subject performed by one or more measurement modules (115), healthcare data of the subject provided by one or more personal devices (135) of the subject, and anamnestic data of the subject, and provide an outcome of the survey based on said metabolic condition and said correlation.

### CROSS REFERENCE TO RELATED APPLICATIONS AND INCORPORATION BY REFERENCE

This application claims priority from commonly owned PCT Patent Application PCT/EP2024/062130 filed 2 May 2024 and titled “SYSTEM FOR SURVEYING THE HEALTH AND/OR PERFORMANCE STATE OF A SUBJECT”, and from commonly owned Italian Patent Application 102023000008940 filed 5 May 2023 and titled “SYSTEM FOR SURVEYING THE HEALTH AND/OR PERFORMANCE STATE OF A SUBJECT”. This application also incorporates by this reference the entirety of PCT Patent Application PCT/EP2024/062130, and the entirety of Italian Patent Application 102023000008940.

### BACKGROUND ART

### Technical Field

The present invention relates to a system for surveys about the health and/or performance state of a living subject, for example of a human being. Particularly, the present invention relates to a system for determining a metabolic condition of the subject (for example, presence or absence of alterations in metabolism, or “dysmetabolism”, of the subject or of one or more zones of his/her body) and/or a correlation of the metabolic condition with a plurality of information concerning, or associatable with, a general or specific health and/or performance state of the subject.

### Overview of the Related Art

The metabolism of an organism (a living subject) it is the set of biochemical reactions, and alterations in metabolism (“dysmetabolism”) of the subject (or of one or more zones of his/her body) may have correlations with ongoing symptoms and/or pathologies in the subject and/or with predispositions of the subject to develop certain pathologies. Just as an example, metabolic alterations may be present in case of tumors. Just as another example, in applications in the field of the sports medicine, the metabolism of a subject may provide information about methods for optimizing athletic performance, preventing injuries and accelerating recovery following injury.

The human body emits electromagnetic fields, and it is possible to measure one or more bioelectric quantities associated with such emitted electromagnetic fields (such as electric voltage, electric current, electric impedance).

The measurements of the bioelectric quantities associated with the human body are typically affected by conditions/parameters such as acquisition points of the measurements, measurement conditions (for example, electric and/or geometric features of measurement electrodes, pressure exerted by the measurement electrodes on the body surface of the subject, humidity of the body surface of the subject), possible movements of the subject during the measurements, and emotional and physiological conditions of the subject during the measurements. Electrocardiogram and electroencephalogram are examples of the issues that are encountered during the measurements of the bioelectric quantities associated with the body of a living subject.

Scientific literature exists which relates to the investigation of bioelectric behavior of the human body and the electrical activities of the human body measurable in certain points of the body.

Just as an example, paper “Review of Evidence Suggesting That the Fascia Network Could Be the Anatomical Basis for Acupoints and Meridians in the Human Body” by Yu Bai, Jun Wang, Jin-peng Wu, Jing-xing Dai, Ou Sha, David Tai Wai Yew, Lin Yuan and Qiu-ni Liang, published on 26 Apr. 2011, discloses a study about the anatomical grounds for the concept of meridians in Traditional Chinese medicine.

Just as another example, paper “Meridian is a Three-Dimensional Network from Bio-Electromagnetic Radiation Interference: An Interference Hypothesis of Meridian” by Jinxiang Han, published on 30 Sep. 2011, analyzes the biological electromagnetic radiation of the human body.

Just as a further example, paper “Telocytes in Different Organs of Vertebrates: Potential Essence Cells of the Meridian in Chinese Traditional Medicine” by Shi Yonghong, Wu Ruizhi, Zhang Yue, Bai Xuebing, Imran Tarique, Liang Chunhua, Yang Ping and Chen Qiusheng—MOE Joint International Research laboratory of Animal Health and Food Safety, College of Veterinary Medicine, Nanjing Agricultural University, Nanjing, Jiangsu—Province 210095, China, published on 26 Jun. 2020, points out a physical correspondence between meridians and molecular infrastructure, which forms a dense and branched network with electrical functions.

The use of artificial intelligence engines (or algorithms) (for example, artificial intelligence engines implementing autonomous learning functionalities (“Machine Learning”)) in the medicine application domain dates back to the mid-twentieth century, but only in recent periods there has been a transfer of academic knowledge into healthcare solutions.

The increase in quantity and complexity of data available for patients in the routine health care, makes alternative approaches necessary for the corresponding processing.

Artificial intelligence engines, and particularly artificial intelligence engines implementing autonomous learning functionalities, are being used to an increasing extent in medical applications for the identification of prediction models and the recognition of deep-learning schemes and techniques (the “deep-learning” is a branch of the autonomous learning comprising techniques based on layered artificial neural networks), in order to combine large amounts of complex data, find correlations and meanings, and support human personnel in performing tasks that, otherwise, would require considerable time and experience (and which could in any case give rise to assessment errors).

### SUMMARY OF THE INVENTION

The Applicant has found that, despite the knowledge currently available exposed in the previous section of this description, there are aspects still not investigated about the use of the information deriving from the electromagnetic behavior of the body of a living subject (for example, of a human body) by applying analytical functionalities (for example, based on comparisons of large amounts of data and/or on artificial intelligence engines).

The present invention relates to a system as defined in the annexed independent claims, with optional, facultative features that are defined in the dependent claims.

Particularly, the present invention relates to a system for submitting a subject to a survey aimed at assessing a health state and/or a performance state of the subject.

The system comprises one or more biosensors adapted to detect, in one or more points of the body surface, a plurality of bioelectric signals emitted by the body of the subject and associated with at least one body zone dependent on a purpose of the survey.

The system comprises an acquisition module configured to acquire said plurality of bioelectric signals.

The system comprises one or more substances to which the subject has to be exposed during the execution of the survey. The one or more substances are configured for inducing, by exposure of the subject thereto, reactions in one or more molecules that are involved in one or more biochemical reactions that constitute a metabolic chain in the at least one body zone.

The system comprises an analysis apparatus configured to implement one or more mathematical algorithms to:
determine a metabolic condition of the subject according to a difference between first bioelectric signals, among said plurality of bioelectric signals, spontaneously emitted by the body of the subject in the absence of exposure of the subject to said one or more substances, and second bioelectric signals, among said plurality of bioelectric signals, emitted by the body of the subject in response to the exposure of the subject to said one or more substances. The exposure of the subject to said one or more substances comprises the placement of the one or more substances externally to the body of the subject and in proximity or contact with the body of the subject. In presence of an altered metabolic condition the second bioelectric signals are different from the first bioelectric signals, as a result of said inducing reactions in said one or more molecules;
identify a correlation between the metabolic condition of the subject and at least one among:
   nutritional data and/or sport medicine data;
   statistical data associated with one or more pathologies;
   diagnostic/clinical data of the subject obtained through one or more medical devices;
   measurements of vital signs of the subject performed by one or more measurement modules;
   healthcare data of the subject provided by one or more personal devices of the subject, and
   anamnestic data of the subject, and
provide an outcome of the survey based on said metabolic condition and said correlation.

According to an embodiment, the analysis apparatus comprises an artificial intelligence engine configured to operate based on a plurality of training data relating to a population of subjects. According to an embodiment, the analysis apparatus is configured to determine said metabolic condition and/or identify said correlation based on a similarity of the subject to other subjects in the population of subjects.

According to an embodiment, the analysis apparatus is configured to identify possible undesired electrical contributions provided to said plurality of bioelectric signals by one or more body zones of the subject different from said at least one body zone, and compensate said plurality of bioelectric signals according to said undesired electrical contributions. According to an embodiment, the analysis apparatus is configured to determine said metabolic condition of the subject and/or identify said correlation according to said plurality of compensated electrical signals.

According to an embodiment, the analysis apparatus is configured to determine a validity and/or quality of the plurality of bioelectric signals.

According to an embodiment, the analysis apparatus is configured to perform a pre-processing of the plurality of bioelectric signals for determining at least one bioelectric emission parameter. According to an embodiment, the analysis apparatus is configured to determine said metabolic condition of the subject and/or identify said correlation according to said at least one bioelectric emission parameter.

According to an embodiment, said at least one bioelectric emission parameter comprises one or more among:
an average bioelectric emission index;
a total bioelectric emission index;
statistical variance and/or standard deviation of the plurality of bioelectric signals;
relative differences between bioelectric emissions associated with different body zones of said at least one body zone of the subject;
a response index indicative of an impact of at least one of said one or more external stimuli on at least one of the at least one body zone;
a relative comparison between sub-sets of the acquired bioelectric signals.

According to an embodiment, the system comprises one or more external stimuli sources each one adapted to generate one or more external stimuli to which the subject has to be exposed during the execution of the survey. According to an embodiment, the second bioelectric signals are emitted by the body of the subject further in response to the exposure of the subject to at least one of said one or more external stimuli.

According to an embodiment, said external stimuli sources comprise at least one among:
one or more acoustic stimulus generators;
one or more luminous and/or/visual stimulus generators, and
one or more olfactory stimulus generators.

According to an embodiment, each biosensor comprises or incorporates said one or more substances.

According to an embodiment, the system comprises said one or more medical devices and/or said one or more measurement modules.

According to an embodiment, said medical devices comprise one or more among:
an ultrasound machine;
an electrocardiograph;
an electroencephalograph;
a blood analysis apparatus;
a urinalysis device;
a stool analysis device.

As better discussed in the detailed description of exemplary embodiments, the system according to the present invention allows submitting a subject (for example, a living human being) to a survey (or screening or investigation) aimed at evaluating the health and/or performance state of the subject, wherein the survey comprises a metabolic survey aimed at tracing and evaluating, in a quick, non-invasive and accurate manner, a metabolic condition of the subject based on a state of one or more metabolic chains in one or more organs of the body of the subject (for example, in order to identify dysmetabolism, such as a metabolic chain that exhibits an abnormal request of specific substances used and/or produced by the organism), and wherein, depending on a purpose of the survey, the metabolic condition may be correlated with proper data. Examples of purposes of the survey comprise, but are not limited to: defining training programs and/or nutritional/food plans; supporting evaluations and psychological programs; allowing early diagnoses; identifying ongoing pathologies; understanding or identifying the metabolic causes underlying specific symptoms manifested by the subject, and determining the metabolic causes of anomalies resulting from diagnostic/clinical exams.

### BRIEF DESCRIPTION OF THE FIGURES

Features and advantages of the present invention set out in the previous section, as well as other features and advantages, will be apparent by reading the following detailed description of exemplary and non-limiting possible embodiments thereof. The following description will be better understood by making reference, during reading, to the annexed drawings, wherein:

FIG. 1 shows, in terms of functional blocks, a system according to embodiments of the present invention, and

FIG. 2 shows a simplified structure of a survey protocol that may be implemented by the system of FIG. 1, according to embodiments of the present invention.

### DETAILED DESCRIPTION OF EXEMPLARY EMBODIMENTS OF THE PRESENT INVENTION

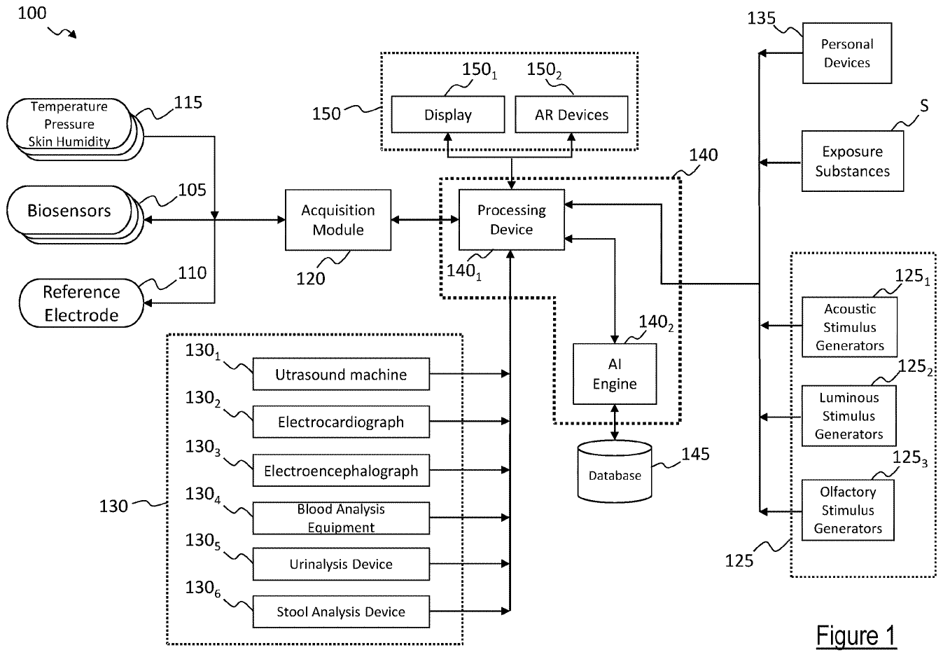

With reference to the figures, FIG. 1 shows, in terms of functional blocks, a system 100 according to embodiments of the present invention.

In the following, when one or more features of the system 100 are introduced by the wording “according to an embodiment”, they are to be construed as features additional or alternative to any features previously introduced, unless contrary indicated and/or unless there is an apparent incompatibility among feature combinations that is immediately apparent to the skilled person.

According to an embodiment, the system 100 is adapted to submit a subject (for example, a living human being) to a survey aimed at evaluating the health and/or performance state of the subject.

The survey comprises a metabolic survey. For the purposes of the present description, a metabolic survey represents a survey capable of determining a metabolic condition (or trace a metabolic profile) of the subject in one or more zones of the body (or body zones) of the subject (for example, one or more organs and/or one or more tissues and/or one or more body parts of the subject), particularly by determining excesses or deficiencies of certain substances (for example, enzymes) in order to identify metabolic imbalances (or dysmetabolism) in one or more body zones of the subject (as discussed in the following).

For the purposes of the present description, the metabolic condition or metabolic profile in one or more body zones of the subject indicates the state of the succession of the biochemical reactions (or of a part thereof) that constitute, as a whole, the metabolic chain occurring in these body zones for converting molecules from one form to another. An abnormal state of one or more of such biochemical reactions identifies an altered or unbalanced metabolic condition, or dysmetabolism.

According to an embodiment, the body zone(s) where the metabolic survey is adapted to trace the metabolic profile depends on a purpose of the survey. Examples of purposes of the survey comprise, but are not limited to: identifying dysmetabolism; defining training programs and/or nutritional/food plans; supporting evaluations and psychological programs; allowing early diagnoses; identifying ongoing pathologies; understanding or identifying the metabolic causes underlying specific symptoms manifested by the subject, and determining the metabolic causes of anomalies resulting from diagnostic/clinical exams. As better discussed in the following, the metabolic condition determined by the metabolic survey is used, for example by correlation of the metabolic condition with proper data depending on the purpose of the survey, to provide an outcome of the survey, for example nutritional/food plans (for example, diets and/or food integration programs), and/or training programs, and/or evaluations and psychological programs, and/or early diagnoses and/or risk factors for the subject to develop one or more pathologies (the term pathology denoting, in the context of the present description, any diseases, syndromes, disturbs, disorders, or disfunctions of the human body), and/or interpretation or explanation of diagnostic/clinical exams.

As better discussed in the following, an interesting (although not exclusive) application has the metabolic survey intended to integrate or be integrated by diagnostic and/or clinical exams (in the following, diagnostic/clinical exams) performed through one or more conventional medical apparatuses, for example in order to understand or identify the metabolic causes underlying specific symptoms manifested by the subject and/or determine the metabolic causes of anomalies resulting by the diagnostic/clinical exams (so as to implement targeted, customized and effective therapies), and/or identify ongoing pathologies and/or identify risk factors for the subject to develop one or more pathologies.

In the following, a survey in which the metabolic survey is intended to integrate or be integrated by diagnostic/clinical exams will be concisely referred to as diagnostic/clinical survey.

Examples of diagnostic/clinical surveys comprise, but are not limited to, cardiovascular surveys, cardiorespiratory surveys, oncologic surveys, mental health surveys, psychological surveys, neurodegenerative surveys, endocrinology surveys, gastrointestinal surveys, sport and stress load resistance surveys, and stress resilience surveys.

Without losing generality, the diagnostic/clinical exams may be provided in any suitable forms, such as texts, images or a combination thereof.

According to an embodiment, the system 100 comprises one or more biosensors 105 adapted to detect, for example in one or more points of the body surface (in the following, tie points), bioelectric signals emitted by the body of the subject.

According to an embodiment, as discussed in the following, the tie points are associated with one or more body zones. As mentioned in the foregoing, the body zone(s) depend on the purpose of the survey. In case of diagnostical/clinical survey, the body zone(s) for example depend on the type of diagnostic/clinical survey. Without losing generality, depending on the purpose of survey and/or on the anatomy of the body zones under investigation or consideration (i.e., the body zones involved in the survey), the tie points may be points of the body surface that are in correspondence of the body zones under investigation and/or points of the body surface that are far or relatively far from the body zones under investigation.

According to an embodiment, each biosensor 105 may comprise one or more electrodes.

According to an embodiment, each biosensor 105 may comprise a plurality of micro-electrodes (for example, in order to build a bioelectric signal emission map corresponding to the tie points covered by the micro-electrodes).

According to an embodiment, each biosensor 105 may be shaped as a wearable device, which may be worn by the subject. This allows facilitating the monitoring of the subject in cases where he/she is moving (for example, while practicing sports). Just as a non-limitative example, each biosensor 105 may comprise a patch or plaster or bandage for allowing wearability thereof, the electrodes or the micro-electrodes being for example integrated in the patch. Just as another non-limitative example, each biosensor 105 may be shaped as glove, sock, or thimble, so as to detect the bioelectric signals emitted by body of the subject in correspondence of hands or feet.

According to an embodiment, each biosensor 105 may be shaped as a pen probe (for example, with a detection electrode for detecting the bioelectric signal that is in correspondence of a tip of the probe). According to an embodiment, each pen probe may comprise a mechanical adjusting device (for example, a spring) adapted to mechanically adjust (i.e., control and stabilize) the contact pressure of the electrode with the body surface of the subject (so as to apply the appropriate pressure on the body surface, substantially regardless of the pressure exerted by the operator).

According to an embodiment, each biosensor 105 may comprise or incorporate exposure substances (discussed in the following).

According to an embodiment, the system 100 comprises a reference (or ground) electrode 110.

For the purposes of the present invention, the reference electrode 110 is adapted to provide a reference signal for the correct acquisition of the bioelectric signals.

Just as a non-limitative example, the reference electrode 110 may be placed in correspondence of the wrist of the subject.

According to an embodiment, the reference electrode 110 may be shaped as a wearable device (for example, similarly or substantially similarly to the above discussion for the biosensors 105).

The reference electrode 110 may be omitted in basic embodiments of the present invention. In such embodiments, the reference electrode 110 may be replaced by circuit solutions implemented, for example, in an acquisition module of the bioelectric signals (discussed in the following).

According to an embodiment, the system 100 comprises one or more measurement modules 115 adapted to measure one or more vital parameters of the subject (particularly, current vital parameters, i.e., vital parameters measured during the execution of the survey).

Examples of vital parameters comprise, but are not limited to, blood pressure, skin humidity level, and body temperature. According to embodiments, the measurement modules 115 may comprise one or more among a blood pressure monitor, a skin humidity monitor, and a body temperature monitor.

Without losing generality, the presence and/or the use of the measurement modules 115 (or of a subset thereof) may depend on the survey for which the system 100 is conceived and/or on the purpose of the survey (and, hence, on the need of one or more vital parameters for performing such survey). Particularly, the measurement modules 115 (or at least a subset thereof) may be omitted in basic embodiments of the present invention.

According to an embodiment, the system 100 comprises an acquisition module 120 According to an embodiment, the acquisition module 120 is configured to acquire the bioelectric signals (i.e., the bioelectric signals detected by the biosensors 105).

Without losing generality, the acquisition module 120 may be configured to acquire the bioelectric signals in forms of electric currents or electric voltages.

According to an embodiment, the acquisition module 120 is configured to perform one or more processing of the bioelectric signals thereby obtaining corresponding data (in the following generically denoted as bioelectric data).

According to an embodiment, bioelectric signal processing comprises, but is not limited to, sampling and/or filtering and/or compensations and/or suppressions/rejections of the bioelectric signals (as discussed here below), and is based on one or more mathematical algorithms (such as computational models) implemented by the acquisition module 120.

Just as an example, the acquisition module 120 may be configured to compensate for changes in the detections of the bioelectric signals due to changes (for example, significant and/or quick changes) in data acquisition parameters (e.g., due to the different contact pressures of the electrodes on the body surface of the subject, such as due to different pressures exerted by the operator on different electrodes) or in the vital parameters (i.e., blood pressure and/or skin humidity level and/or body temperature), the bioelectric data provided by the acquisition module 120 comprising for example such compensations.

Just as another example, the acquisition module 120 may be configured to normalize the detections of the bioelectric signals with respect to material and/or shape and/or size of the biosensors 105 (indeed, material and/or shape and/or size of the biosensors 105 may provide an additional electrical potential contribution being additional with respect to the one associated with the natural emission of the body of the subject).

Just as a further example, the acquisition module 120 may be configured to suppress/reject abnormal bioelectric signals and/or non-consistent bioelectric signals. Examples of abnormal bioelectric signals comprise, but are not limited to, bioelectric signals with slow or relatively slow signal response curves (for example, signal response curves higher than 4-5 seconds or other time interval, i.e., bioelectric signals with transients longer than such time interval). Without losing generality, the acquisition module 120 may be configured to recognize the abnormal bioelectric signals by real-time comparison of the (acquired) bioelectric signals with proper reference signal response curves (the reference signal response curves depending for example on the tie points where the biosensors 105 are applied for detection and/or on the body zone(s) under investigation). Examples of non-consistent bioelectric signals comprise, but are not limited to, bioelectric signals indicative of measurement unbalances with respect to previous measurements and/or with respect to reference measurements, and bioelectric signals acquired in symmetrical tie points of the body but exhibiting unexpectedly different values and/or shapes. In case of abnormal bioelectric signals and/or non-consistent bioelectric signals, the acquisition module 120 may be configured to perform or solicit new bioelectric signal acquisitions.

According to an embodiment, the acquisition module 120 may be shaped as electronic board, electronic device or electronic apparatus.

According to an embodiment, the acquisition module 120 is separate with respect to the biosensors 105.

According to an embodiment, the acquisition module 120 is coupled to the biosensors 105 (for receiving the corresponding bioelectric signals detected by them), to the reference electrode 110 (when provided, for receiving the corresponding reference signal), and to the measurement modules 115 (when provided, for receiving the measurements of the corresponding vital parameters).

According to an embodiment, the acquisition module 120 may be coupled to the biosensors 105 (or to a subset thereof) and/or to the reference electrode 110 and/or to the measurement modules 115 (or to a subset thereof) through respective wired links. Additionally or alternatively, the acquisition module 120 may be coupled to the biosensors 105 (or to a subset thereof) and/or to the reference electrode 110 and/or to the measurement modules 115 (or to a subset thereof) through respective wireless links, for example short-range wireless links (for example, Bluetooth™ links).

According to alternative embodiments, not shown, the acquisition module 120 may be integrated in the biosensors 105.

The system 100 comprises one or more substances S (hereinafter, exposure substances) to which the subject has to be exposed during the execution of the survey. As better discussed here below, the exposure substances S are configured for inducing, by exposure of the subject thereto, reactions in one or more molecules, hereinafter also referred to as substance-responsive molecule(s), that are involved in one or more biochemical reactions that constitute one or more metabolic chains in body zone(s) under investigation. Such reactions are temporary, in that they take place for a short or relatively short time interval after or upon exposition of the subject to the exposure substance(s) (typically, such reactions take place for a time interval significantly shorter than the exposition time of the subject to the exposure substance(s)).

According to an embodiment, the system 100 comprises one or more external stimuli sources 125 each one adapted to generate one or more respective external stimuli to which the subject may be exposed (in addition to the exposure to the exposure substances) during the execution of the survey. According to an embodiment, the external stimuli are external sensory stimuli.

As will be understood in the following of the present description, the system 100 is configured to perform the metabolic survey (i.e., determine the metabolic condition of the subject) based on possible differences between the bioelectric signals (in the following, basal bioelectric signals) spontaneously emitted by the body of the subject in absence of exposure of the subject to the exposure substances S, and the bioelectric signals (in the following, induced bioelectric signals) emitted by the body of the subject in response to the exposure of the subject to the exposure substances S (and, possibly, also to the exposure to one or more external stimuli produced by one or more of the external stimuli sources 125). Since in a tie point the human body responds to the presence of an external exposure substance, and possibly of an external sensory stimulus, with an increase, a decrease or an absence of a change in the bioelectric signal with respect to the basal bioelectric signal, the type of response is indicative of the condition of the body zone(s) associated with such tie point.

The exposure substances S are configured to interact with the body of the subject, by inducing reactions in it that are detectable through temporary changes in the bioelectric signals with respect to the basal bioelectric signal. Particularly, for the purposes of the present description, an exposure substance is a substance that, through interaction (by exposure, as discussed in the following) with the body zone(s) under investigation, induces, in presence of metabolic anomalies, a corresponding physiological reaction, and particularly a response or alteration in one or more respective exposure substance-responsive molecules (e.g., enzymes and coenzymes) involved in one or more of the biochemical reactions that constitute the metabolic chain in these body zone(s). The response of the exposure substance-responsive molecule(s) induced by the exposure substances results, in presence of metabolic anomalies or dysmetabolism, in corresponding bioelectric signals different from the respective basal bioelectric signals (the higher the level of dysmetabolism, the higher the difference between the bioelectric signals and the respective basal bioelectric signals). In other words, in presence of dysmetabolism, the interaction between the body zone(s) and the exposure substance generates a response in one or more of the biochemical reactions of the metabolic chain (for example, a higher “metabolic request” of the exposure substance in case of deficiencies of such a substance in the metabolic chain), which response is detected as bioelectric signals different from the respective basal bioelectric signals, whereas in absence of dysmetabolism the interaction between the body zone(s) and the exposure substance generates no or substantially no response in the biochemical reactions of the metabolic chain (for example, no “metabolic request” of the exposure substance), which response is detected as bioelectric signals equal or substantially equal to the respective basal bioelectric signals.

Thus, the metabolic condition so determined provides in-depth information about the subject (particularly, the body zone(s) under investigation) as compared to conventional diagnostical/clinical exams. Just as an example, whereas a blood test is capable of detecting an absolute quantity of a certain molecule that could fall within a statistical range of normality compared to the average population, the metabolic condition determined by exposure to the subject to the exposure substances provides specific information about the extent of the metabolic request of the molecule (in that it allows recognizing a higher metabolic request of the molecule in specific organs, and hence it allows recognizing a “distribution” of this molecule, and possible unbalances in the distribution, which could not be derived by the value of overall quantity of the molecule provided by the blood test).

Without losing generality, the exposure substances S may be or comprise the same exposure substance-responsive molecules (for example, in suitable dilution). In this case, as mentioned in the above example, the response generated by the exposure substances S in one or more of the biochemical reactions of the metabolic chain may correspond to a higher “metabolic request” of the exposure substance in case of its deficiencies in the metabolic chain.

Without losing generality, the exposure substances may be or comprise one or more molecules different from the exposure substance-responsive molecule, for example antagonist molecules being antagonist to the exposure substance-responsive molecule.

The exposure of the subject to the exposure substances, which determines said interaction between the exposure substance and the body zone(s) under investigation, comprises the placement (e.g., at a proper phase of the survey) of the exposure substances externally to the body of the subject, for example in contact with, or in proximity of, the body zone(s) under investigation. More generally, the exposure of the subject to the exposure substances comprises the placement (e.g., at a proper phase of the survey) of the exposure substances externally to the body of the subject, at a distance that allows interaction, at electromagnetic field level, between the stimulus substances and the human body, in particular the exposure substance-responsive molecules in the body zone(s) under investigation.

According to an embodiment, the exposure substances S may be contained in respective vials/test tubes to be placed in contact with, or in proximity of, the body of the subject at a proper phase of the survey. Without losing generality, the contact of the vials/test tubes with the body of the subject may be direct or indirect (for example, through electrical conductors, such as copper conductors, and/or dedicated electronic circuits).

According to an embodiment, the exposure substances S may be incorporated (at least in part) in the biosensors 105 (or at least in a subset thereof).

Without losing generality, the exposure substances S may comprise a single type of molecule or a plurality of types of molecules, for example depending on number and types of reactions of the body of the subject under evaluation.

Without losing generality, the exposure substances S may comprise any substances (for example a selected protein) that, for example based on medical literature, is involved in the human metabolism, and particularly in the metabolic chain of the body zone(s) under investigation for the survey.

Just as a non-limitative example, for an oncologic survey the exposure substances S may comprise C-MYC, C-RAS, PchoCl, Petn, Caspases-3, Caspases-7 (or a set of others molecules permitting to better distinguish specific types of tumor)).

Just as another non-limitative example, for a mental health survey (for example, for identifying dysmetabolism in subjects suffering from Alzheimer) the exposure substances S may comprise beta Amyloid, Caspase-7, Ig-A, Ig-G, Methylenedioxyamphetamine (MDA) (or a subset thereof).

Just as a further non-limitative example, for a stress resilience survey (for example, for identifying a tendency to emotional instability in subjects performing work tasks requiring concentration, short response times, stress, emotional control) the exposure substances S may comprise adrenaline, norepinephrine, glutamate, aspartate (or a subset thereof). Just as a further non-limitative example, for a psychological survey (for example, for identifying depression states, catatonia, anxiety or panic), the exposure substances S may comprise serotonin, GABA, cortisol or adrenaline, respectively.

According to an embodiment, the external stimuli sources 125 may comprise one or more acoustic stimulus generators 125₁.

According to an embodiment, the external stimuli sources 125 may comprise one or more luminous and/or visual stimulus generators 125₂.

According to an embodiment, the external stimuli sources 125 may comprise one or more olfactory stimulus generators 125₃.

Without losing generality, the presence and/or the use of the external stimuli sources 125 (or of a subset thereof) may depend on the survey for which the system 100 is conceived and/or on the purpose of the survey (and, hence, on the specific external stimuli necessary to the execution of such survey). Particularly, one or more of the external stimuli sources 125 may be omitted in basic embodiments of the present invention. Additionally or alternatively, further external stimuli (such as predefined movements of the body of the subject or of parts thereof, and/or pressures exerted in specific regions of the body of the subject and/or physical exercises, including reading and/or writing, and/or psychological tests) may be provided, for example depending on the purpose of survey and of the body zones involved in the survey.

According to an embodiment, the indication of the exposure substances S and of possible external stimuli to which the subject has to be exposed and their application modes are defined in proper survey protocols (discussed in greater detail in the following).

According to an embodiment, as mentioned in the foregoing, the metabolic survey performed by the system 100 may support (i.e., integrate) or be supported by (i.e., be integrated by) diagnostic/clinical exams of the subject. Examples of diagnostic/clinical exams comprise, but are not limited to, blood analysis, urine analysis, cardiovascular apparatus exams, gastrointestinal apparatus exams, and respiratory tract exams.

According to an embodiment, the system 100 comprises (or is configured to interact with) one or more medical apparatuses 130 adapted to perform respective diagnostic/clinical exams and to provide corresponding diagnostic/clinical data.

Without losing generality, the medical apparatuses 130 (or at least a subset thereof) may be internal or external to the system 100.

According to an embodiment, such medical apparatuses 130 comprise one or more among an ultrasound machine 130₁ (in which case the diagnostic/clinical data comprises for example ultrasound signals), an electrocardiograph 130₂ (in which case the diagnostic/clinical data comprises for example electrocardiographic signals), an electroencephalograph 130₃ (in which case the diagnostic/clinical data comprises for example electroencephalographic signals), a blood analysis equipment 130₄ (in which case the diagnostic/clinical data comprises for example blood values), an urinalysis device 130₅ (in which case the diagnostic/clinical data comprises for example urine values), and a stool analysis device 130₆ (in which case the diagnostic/clinical data comprises for example stool values).

Additionally or alternatively, the medical apparatuses 130 may comprise imaging devices (such as X-ray machines, CT Scanner machines, MRI machines or PET machines), not shown.

Without losing generality, regardless of the specific medical apparatus, the diagnostic/clinical data may be in the form of texts, images or combinations thereof. Without losing generality, the presence and/or the use of the medical apparatuses 130 (or of a subset thereof) may depend on the diagnostic/clinical surveys for which the system 100 is conceived and/or on the selected diagnostic/clinical survey (and, hence, on the specific diagnostic/clinical exams necessary to the execution of such surveys). Particularly, the medical apparatuses 130 (or at least a subset thereof) may be omitted in basic embodiments of the present invention.

According to an embodiment, the system 100 comprises (or is adapted to interact with) one or more personal devices 135 adapted to provide one or more data about the subject.

According to an embodiment, the personal devices 135 may comprise personal devices with healthcare functionalities, i.e. personal devices adapted to provide one or more healthcare data of the subject. Examples of healthcare data comprise, but are not limited to, weight, height, gender, level of motor activity, and medical history. Without losing generality, the healthcare data may comprise current and/or historical healthcare data. Without losing generality, the healthcare data may comprise healthcare data measured by exploiting one or more functionalities of the personal devices 135 and/or healthcare data acquired by the personal devices 135 (for example, manually input or loaded by the subject, or automatically input or loaded into the personal devices 135 through interfacing with specific measurement apparatuses, such as smart scales and/or smart oximeters and/or smart blood pressure monitors and/or smart physical activity monitoring bracelets).

According to an embodiment, such personal devices 135 comprise a smartwatch and/or a smartphone.

Without losing generality, the personal devices 135 (or at least a subset thereof) may be internal or external to the system 100.

Without losing generality, the presence and/or the use of the personal devices 135 may depend on the specific survey(s) for which the system 100 is conceived and/or on the purpose of the survey (and, hence, on the need of one or more healthcare data for the execution of such survey). Particularly, the presence of the personal devices 135 (or at least of a subset thereof) or the possibility of interacting with them, may be omitted in basic embodiments of the present invention.

According to an embodiment, the system 100 comprises an analysis apparatus 140.

According to an embodiment, the analysis apparatus 140 is configured to receive exposure substance data indicative of the exposure substance(s) to which the subject has been exposed (or has to be exposed) and exposure modes (for example, with contact or without contact with the body of the subject, or exposure times).

According to an embodiment, the analysis apparatus 140 is coupled or coupleable to the stimulus sources 125 for receiving corresponding stimulus data indicative of the external stimuli to which the subject has been exposed and application modes thereof (for example, order or sequence of application of the stimuli and/or intensity of application of the stimuli).

According to an embodiment, the analysis apparatus 140 is coupled or coupleable to the acquisition module 120 for receiving the corresponding bioelectric data. As discussed in the following, the analysis apparatus 140 is configured to determine the metabolic condition according to the bioelectric data and the stimulus data (for example in order to identify dysmetabolism, that is, to determinate excesses US Pat or deficiencies of certain substances or unbalancing of a set of molecules), and, depending on the purpose of the survey, to identify a correlation between the metabolic condition and one or more among:
the diagnostic/clinical data (as discussed in the following);
nutritional data. Without losing generality, the nutritional data may comprise one or more among data about the interaction between lifestyle, physical activity, diet and human health, data about the physiological aspects and the molecular mechanisms related to the prevention of diseases with a nutritional component, and data about maintaining a well-being state;
sports medicine data. Without losing generality, the sports medicine data may comprise one or more among data about the biology of the sport and of the reactions of the human body during sport activity by the anthropologic, physiologic and clinic viewpoints, data about the types of sports that can be performed by considering the constitutional qualities, and data about the approaches for the motor rehabilitation of the injured sportsmen or sportswomen;
statistical data associated with one or more pathologies. Without losing generality, the statistical data may comprise data associated with one or more pathologies, resulting from observational (prospective and/or retrospective) studies and/or clinical trials;
the measurements of the vital parameters of the subject;
the healthcare data of the subject;
anamnestic data of the subject, i.e., information about the subject's medical history, and particularly subject's current health concerns, past illnesses, surgeries, medications, lifestyle habits, and family medical history (the anamnestic data being for example gathered through questioning and interview).

According to an embodiment, the analysis apparatus 140 is coupled or coupleable to the medical apparatuses 130 (or to a subset thereof) for receiving the corresponding diagnostic/clinical data.

Without losing generality, the analysis apparatus 140 is configured to receive the diagnostic/clinical data (or a subset thereof) directly (for example, through a proper wired or wireless communication channel between the analysis apparatus 140 and the medical apparatuses 130) or indirectly (for example, through manual or automatic US Pat acquisition of the diagnostic/clinical data in digital format).

As discussed in the following, the analysis apparatus 140 (o, more generally, the system 100) is configured to perform diagnostic/clinical surveys according to the bioelectric data, the exposure substance data, the stimulus data (when provided), and the diagnostic/clinical data.

According to an embodiment, the analysis apparatus 140 comprises a processing device 1401.

According to an embodiment, the processing device 1401 is configured to perform a software application (in the following, survey software).

According to an embodiment, the survey software provides a user interface adapted to allow the interaction with a user of the system 100.

According to an embodiment, the survey software provides a system interface adapted to handle data transfer through the system 100. Without losing generality, the system interface is adapted to allow interaction of the analysis apparatus 140 with the acquisition module 120, the exposure substances S, the stimulus sources 125 (particularly, the acoustic stimulus generators 125₁, when provided, the luminous and/or visual stimulus generators 125₂, when provided, and the olfactory stimulus generators 125₃, when provided), the medical apparatuses 130 (when provided), the personal devices 135 (when provided), and display devices (discussed in the following).

According to an embodiment, the survey software is configured to provide a plurality of processing activities (discussed in the following).

Without losing generality, the processing device 140₁ may comprise a local processing device (for example, a personal computer), in which case the survey software may be a local software application, and/or a remote processing device (for example, a remote server of a “Cloud Computing” network), in which case the survey software may be a distributed software application.

In case of hybrid implementation in which the processing device 140₁ comprises a local processing device and a remote processing device, according to embodiments the local processing device may be configured for data transfer to the remote processing device in asynchronous manner (i.e., in real-time or in near real-time), or in a synchronous or regular manner (i.e., at a following time, for example through a batch process).

According to an embodiment, the analysis apparatus 140 comprises an artificial intelligence engine (or algorithm) (or AI (“Artificial Intelligence”) engine) 140₂.

According to an embodiment, the AI engine 140₂ integrates a plurality of mathematical models within it.

According to an embodiment, the AI engine 140₂ integrates autonomous learning functionalities (or “Machine Learning”) within it.

Without losing generality, the AI engine 140₂ may be implemented through one or more software applications residing and run in the processing device 140₁ (the software applications implementing the AI engine 140₂ being for example performed as instances of the survey software), or (as exemplary illustrated) through one or more software applications residing and run in an entity external to the processing device 140₁ and coupled thereto (for example, through wired and/or wireless links).

According to an embodiment, the AI engine 140₂ is configured to operate according to a plurality of training data.

According to an embodiment, depending on the purpose of the survey, the training data may relate to a population of subjects.

Additionally or alternatively, depending on the purpose of the survey, the training data may relate to pathology patterns. For the purposes of the present invention, a pathology pattern is a pattern related to a specific pathology, and refers to a recognizable set of characteristics or features that are consistently associated with a particular disease or condition.

The training data comprises for example off-line training data (for example, provided before the first use of the system 100) and/or on-line training data (for example, acquired during the operation of the system 100 and processed through the autonomous learning functionalities).

According to an embodiment, the AI engine 140₂ is configured to assign the training data to respective categories.

According to an embodiment, the AI engine 140₂ is configured to assign the training data to respective categories according to classification techniques (supervised training), i.e. in which the categories are known a priori, with availability of examples for each category. Without losing generality, the classification may be based on a specific taxonomy, and/or on a formal hierarchy, and/or on a formal ontology (i.e., the formal representation of a domain of interest containing the relevant entities and their relationships in the domain).

According to an embodiment, the AI engine 140₂ is configured to assign the training data to respective categories according to grouping or clustering techniques (non-supervised training), i.e. in which it is assumed that there exists a natural subdivision in categories, without however any a priori knowledge about number and typology of such categories.

Just as a non-limitative example, the training data relating to a population of subjects may be assigned (through classification and/or clustering techniques) to categories such as records of the subjects, anamnestic data of the subjects, records of the doctors, used devices, records functional to the generation of the outcome of the survey (for example, dysmetabolism records, physiological, morphological and anatomical records, bioelectric data, diagnostic/clinical data, exposure substance data, stimulus data, nutritional and/or sports medicine data, statistical data associated with one or more pathologies, healthcare data), parameters characterizing the data acquisition and processing processes: in this way, a subject submitted to survey may be evaluated (other than according to the bioelectric data and the stimulus data, and possibly the nutritional and/or sports medicine and/or statistical and/or the diagnostic/clinical and/or the healthcare data) according to a “similarity” with other subjects of the population of subjects.

According to an embodiment, the training data are stored in a database (discussed in the following) coupled to the AI engine 140₂.

As mentioned in the foregoing, according to an embodiment the survey software (or, more generally, the analysis apparatus 140) is configured to implement, for example by exploiting functionalities of the processing device 140₁ and/or of the AI engine 140₂, a plurality of processing activities involved in the survey.

According to an embodiment, the processing activities comprise check functionalities for checking the bioelectric data acquired by the acquisition module 120, for example in order to determine validity and/or quality of the bioelectric signals (for example, so as to identify possible anomalies in the acquisition of the bioelectric signals). Additionally or alternatively, such check functionalities may be aimed at determining initial and final bioelectric signals of sequences of bioelectric signals acquired in different conditions (for example, in absence of exposure substance(s), in in presence of exposure substance(s), in absence of external stimuli, in presence of external stimuli).

According to an embodiment, the processing activities comprise pre-processing functionalities of the bioelectric signals (and/or of the bioelectric data), for example in order to determine one or more bioelectric emission parameters. Without losing generality, such bioelectric emission parameters may comprise one or more among:
an average bioelectric emission index, for example determined as average calculated on the totality of the basal bioelectric signals acquired from the subject;
a total bioelectric emission index, for example determined as sum calculated on the totality of the bioelectric signals (i.e., in absence and in presence of exposure substances or in absence and in presence of external stimuli) acquired from the subject;
statistical variance and/or standard deviation of the bioelectric signals acquired from the subject;
relative differences between bioelectric emissions associated with different body zones of the subject;
a response index indicative of an impact of one or more of the external stimuli applied on one or more of the body zones of the subject;
a relative comparison between sub-sets of the acquired bioelectric signals.

According to an embodiment, the processing activities comprise analysis functionalities adapted to analyse the bioelectric data, and/or the exposure substance data, and/or the stimulus data, and/or the diagnostic/clinical data, and/or the nutritional and/or sports medicine data, and/or the statistical data, and/or the measurements of the vital parameters, and/or the healthcare data.

According to an embodiment, the analysis functionalities are implemented by the processing device 140₁ and/or through cooperation between the processing device 140₁ and the AI engine 140₂.

According to an embodiment, the analysis functionalities are implemented by means of dedicated mathematical algorithms (such as computational models) being run in the processing device 140₁ and/or in the AI engine 140₂.

According to an embodiment, the analysis functionalities are adapted to determine possible differences between the basal bioelectric signals spontaneously emitted in the tie points by the body of the subject, and the induced bioelectric signals emitted in the same tie points in response to the exposure of the subject to the exposure substance(s) (and, possibly, to one or more external stimuli) so as to determine a metabolic condition of the subject (for example, presence or absence of dysmetabolism in the subject and/or in the body zones submitted to survey). According to an embodiment, the analysis functionalities may be adapted to determine the metabolic condition of the subject according to procedures for neutralizing the daily metabolic trend of the subject.

According to an embodiment, the analysis functionalities are adapted to determine the metabolic condition of the subject (for example, presence/absence of dysmetabolism in the subject) according to the above-mentioned bioelectric emission parameters (or at least a subset thereof).

According to an embodiment, the analysis functionalities are adapted to determine the metabolic condition of the subject based on mathematical modelling of the metabolic chains. Just as an example, the metabolic condition of the subject may be determined based on a comparison between the bioelectric data (for example, a linearly interpolated function of the bioelectric data) and the metabolic chain models associated with the substance-responsive molecules(s) and the body zone(s) which the bioelectric data refer to.

According to an embodiment, the analysis functionalities are adapted to identify possible undesired electrical contributions brought to the (basal and/or induced) bioelectric signals from one or more body zones different from the body zone(s) under investigation, and compensate the bioelectric signals (thereby obtaining corresponding compensated bioelectric signals) according to such undesired electrical contributions (indeed, between a tie point and a body zone there is actually a dominant correspondence, which is not an exclusive component, in that the bioelectric signals acquired in correspondence of the tie point may be affected by electrical contributions from one or more different body zones). According to an embodiment, the analysis functionalities may be adapted to determine the metabolic condition of the subject according to the compensated electric signals.

According to an embodiment, depending on the purpose of the survey, the analysis functionalities are adapted to identify correlations between the metabolic condition of the subject (for example, presence/absence of dysmetabolism in the subject) and one or more external data among the nutritional and/or sports medicine data, the statistical data associated with one or more pathologies, the diagnostic/clinical data of the subject, the measurements of the vital parameters of the subject, and the healthcare data of the subject. Without losing generality, such correlations may be based on mathematical models specific per type or purpose of survey.

According to an embodiment, the analysis functionalities are adapted to determine the correlation between the metabolic condition of the subject and the external data according to the compensated bioelectric signals.

According to an embodiment, the analysis functionalities are adapted to determine the correlation between the metabolic condition of the subject and one or more among the external data based on a similarity of the subject with other subjects of the population of subjects (which, as discussed in the foregoing, may form corresponding training data of the AI engine 140₂).

According to an embodiment, the analysis functionalities are adapted to determine the correlation between the metabolic condition of the subject and one or more among the external data based on a compliance of the bioelectric data with the pathology patterns (which pathology patterns, as discussed in the foregoing, may form corresponding training data of the AI engine 140₂).

According to an embodiment, the analysis functionalities may provide optimizations based on pathology and/or drug therapy constraints (i.e., constraints due to ongoing pathology and/or drug therapy of the subject). Such optimizations may for example result in suggestions of dietary regimes and/or food supplements.

According to an embodiment, the processing activities comprise assistance functionalities adapted to guide the user of the system 100 during one or more phases of the survey. Without losing generality, such assistance functionalities may be adapted to guide the user of the system 100 in the acquisition of the bioelectric signals (for example by suggesting the user about application modes and times to be used for exposing the subject to the exposure substance(s) and/or for applying the external stimuli), and in the following phases of the survey (for example, by dynamically changing the subsequent phases of the survey depending on the information so far acquired).

According to an embodiment, the processing activities comprise report functionalities, for example in order to provide an outcome of the survey.

According to an embodiment, depending on the purpose of the survey, the outcome of the survey may be generated and made available to the user of the system 100 as a visual reporting (for example, in written and/or graphical, or Virtual Reality form).

Additionally or alternatively, depending on the purpose of the survey, the outcome of the survey may be generated and made available as one or more control parameters adapted to control a treatment/care device (not shown), for example so that the treatment/care device may be configured, or pre-configured, with optimal settings for the type of required treatment/care according to the outcome of the survey. Without losing generality, the treatment/care device may be adapted to treat and/or care psychics, physic and/or psychophysical pathologies through electromagnetic and/or visual solicitations. Just as an example, the treatment/care device may comprise a Tecar therapy device.

According to an embodiment, the system 100 may be configured to provide the control parameters (or a subset thereof) directly (for example, through a proper wired or wireless communication channel between the system 100, for example the analysis apparatus 140, and the treatment/care device) or indirectly (for example, through manual setting of the control parameters in the treatment/care device by the user of the system 100).

Without losing generality, depending on the purpose of the survey and regardless of the form in which the outcome of the survey is generated and made available, the outcome of the survey may point out presence or absence of dysmetabolism, and/or provide or allow arranging food nutritional plans (for example, by suggesting dietary regimes/food supplements), provide or allow arranging training programs, and/or indicate risk indices of exposure of the subject to specific pathologies, and/or determine potential responses to (and/or possible collateral effects of) specific treatments (for example, pharmacological treatments or food programs), and/or provide or allow obtaining maps of specific body zones of the subject (for example, neurotransmitter maps aimed at psychological evaluations of the subject), and/or identify or allow identifying the metabolic causes underlying specific symptoms manifested by the subject and/or the metabolic causes of anomalies resulting from diagnostic/clinical exams (as discussed in greater detail in the following), and/or ongoing pathologies.

Just as a non-limitative example, the correlation between the metabolic condition of the subject and the nutritional and/or sports medicine data may allow arranging nutritional plans and/or training plans (or, in general, actions or suggestions associated with well-being achievement or improvement).

Just as another non-limitative example, the correlation between the metabolic condition of the subject and the statistical data associated with one or more pathologies (possibly together with the diagnostic/clinical data, and/or the measurements of the vital parameters and/or the healthcare data) may allow determining a risk factor relating to such pathologies, for example expressed as an index of probability that the metabolic condition of the subject inclines him/her to develop such pathologies.

Particular (although not exclusive) applicative importance of the processing activities (implemented by the survey software, or more generally by the analysis apparatus 140, for example through cooperation between the processing device 140₁ and the AI engine 140₂) have the diagnostic/clinical surveys in which the corresponding diagnostic/clinical exams (performed through one or more conventional medical apparatuses, such as the medical apparatuses 130) integrate or are integrated with one or more metabolic surveys.

Just as a non-limitative example of diagnostic and/or clinical exam integrated by metabolic survey, in case that an ultrasound image reveals a hyper-echoic mass on the kidney of the subject, a metabolic survey performed on the kidney could allow discriminating its nature (for example, dense cyst, tumour, stone or damage from previous injury).

Just as another non-limitative example of diagnostic and/or clinical exam integrated by metabolic survey, in case that the blood analysis reveals a high erythrocyte sedimentation rate, which represents an inflammatory index, a metabolic survey performed over the whole body of the subject (for example, by using exposure substances capable of detecting inflammations, such as C-reactive protein (CRP) and/or Interleukin 2) could allow locating the inflammation, i.e., identify the involved organs.

Just as a further non-limitative example of diagnostic and/or clinical exam integrated by metabolic survey, in case that the subject has generic symptoms detected by electrocardiographic signals (such as extrasystole or arrhythmia), a metabolic survey performed over the whole body of the subject could provide parameters usually detectable only through blood analysis (such as troponin, creatine phosphokinase (CPK), PCR) and/or detect very short half-life molecules (such as adrenaline, norepinephrine, cortisol, which are significant for anxious and depressive states), thereby allowing significant integration of the information provided by the electrocardiographic signals in contexts in which it is not possible to perform laboratory tests or radiological exams.

According to an embodiment, the system 100 comprises a database 145 adapted to store information associated with the operation of the AI engine 140₂ .

Without losing generality, the database 145 may be a local database or a remote database.

According to an embodiment, the database 145 is coupled to the AI engine 140₂ (for example, through wired and/or wireless links), the AI engine 140₂ being for example configured to interact with the database 145 during the operation.

According to an embodiment, the database 145 is based on a hybrid approach SQL-NOSQL.

According to an embodiment, the database 145 it is a structured database, so as to allow the segmentation of the data into mutually independent domains (for security and performance reasons). Just as an example, each domain may contain data about medical centres and/or medical examinations in a respective country.

According to an embodiment, the information in the database 145 comprises, for each domain, the training data of the AI engine 140₂ arranged and differentiated by category (for example, as mentioned in the foregoing, records of the subjects, anamnestic data of the subjects, records of the doctors, used devices, records functional to the generation of the outcome of the survey, parameters characterizing the data acquisition and processing processes).

In basic embodiments of the present invention, the database 145 may be omitted: in such embodiments, the functionalities of the database 145 may be carried out by storing elements of the analysis apparatus 140 (for example, by one or more entities implementing the processing device 1401 and/or by one or more entities implementing the AI engine 140₂).

According to an embodiment, the system 100 comprises one or more display devices 150 coupled to the analysis apparatus 140 (for example, to the processing device 140₁).

Without losing generality, the display devices 150 are adapted to allow the interfacing of the system 100 with its user.

According to an embodiment, such display devices 150 comprise one or more between displays 150₁ and augmented reality (AR) devices 150₂.

According to an embodiment, the display devices 150 may be coupled to the analysis apparatus 140 (for example, to the processing device 140₁) through respective wired links. Additionally or alternatively, the display devices 150 may be coupled to the analysis apparatus 140 (for example, to the processing device 140₁) through respective wireless links, for example short-range wireless links (for example, Bluetooth™ links).

In basic embodiments of the present invention, the display devices 150 may be omitted.

As mentioned in the foregoing, the presence and/or the use of the measurement modules 115, of the external stimuli sources 125, of the medical apparatuses 130 and of the personal devices 135 may depend on the specific survey(s) for which the system 100 is conceived and/or on the purpose of the survey. Therefore, the system 100 provides for a plurality of configurations, each one associated with a specific survey to which it is possible to submit the subject.

Just as a non-limitative example, in a configuration of the system 100 aimed at performing only the metabolic survey, the medical apparatuses 130 may not be provided or used.

Just as another non-limitative example, in a configuration of the system 100 aimed at performing urological survey, the system 100 may comprise, or be adapted to interact with, the ultrasound machine 130₁ and the urinalysis device 130₅. In such configuration, by combining (for example, thanks to the cooperation between the processing device 140₁ and the AI engine 140₂) the information contained in the ultrasound signals and in the urine analysis with the bioelectric data associated with urological system (for example, dysmetabolism indications), it is possible to understand or identify the metabolic causes underlying specific symptoms of the urologic apparatus manifested by the subject (so as to implement targeted and effective therapies), and/or identify indices of risk for the subject to develop specific pathologies or urological dysfunctions.

Just as a further non-limitative example, in a configuration of the system 100 aimed at performing a cardiologic survey, the system 100 may comprise, or be adapted to interact with, the electrocardiograph 130₂ and the blood pressure monitor. In such configuration, by combining (for example, thanks to the cooperation between the processing device 140₁ and the AI engine 140₂) the information contained in the electrocardiographic signals and in the measurements of the blood pressure with the bioelectric data associated with the cardiac system, it is possible to understand or identify the metabolic causes underlying specific symptoms of the cardiac apparatus manifested by the subject (so as to implement targeted and effective therapies) and/or identify indices of risk for the subject to develop specific pathologies or cardiac dysfunctions.

Just as a further non-limitative example, in a configuration of the system 100 aimed at performing a mental health survey, the system 100 may comprise, or be adapted to interact with, the electroencephalograph 130₃. In such configuration, by combining (for example, thanks to the cooperation between the processing device 140₁ and the AI engine 140₂) the information contained in the electroencephalographic signals with the bioelectric data associated with the cerebral cortex, it is possible to understand or identify the metabolic causes underlying specific behavioural symptoms manifested by the subject (so as to implement targeted and effective therapies), and/or identify indices of risk for the subject to develop specific pathologies or mental dysfunctions.

Just as a further non-limitative example, in a configuration of the system 100 aimed at performing a metabolic survey for wellness applications, the system 100 may comprise, or be adapted to interact with, the personal devices 135. In such configuration, by combining (for example, thanks to the cooperation between the processing device 140₁ and the AI engine 140₂) the healthcare data with the bioelectric data, it is possible to obtain, about the health and/or performance state of the subject, evaluation with a certain reliability in shorter times than the times required to the formulation of an accurate sanitary context of the subject.

According to an embodiment, each configuration of the system 100 is associated with a respective survey protocol. Without losing generality, each survey protocol may be a predefined survey protocol, or it may be a survey protocol developed or updated at a following time (for example, based on possible advances and/or discoveries and/or experimentations in the medical field).

According to an embodiment, the survey protocols associated with the available configurations of the system 100 are stored in proper memory locations (for example, accessible by the user of the system 100). Without losing generality, the survey protocols associated with the available configurations of the system 100 may be stored in memory locations of the analysis apparatus 140.

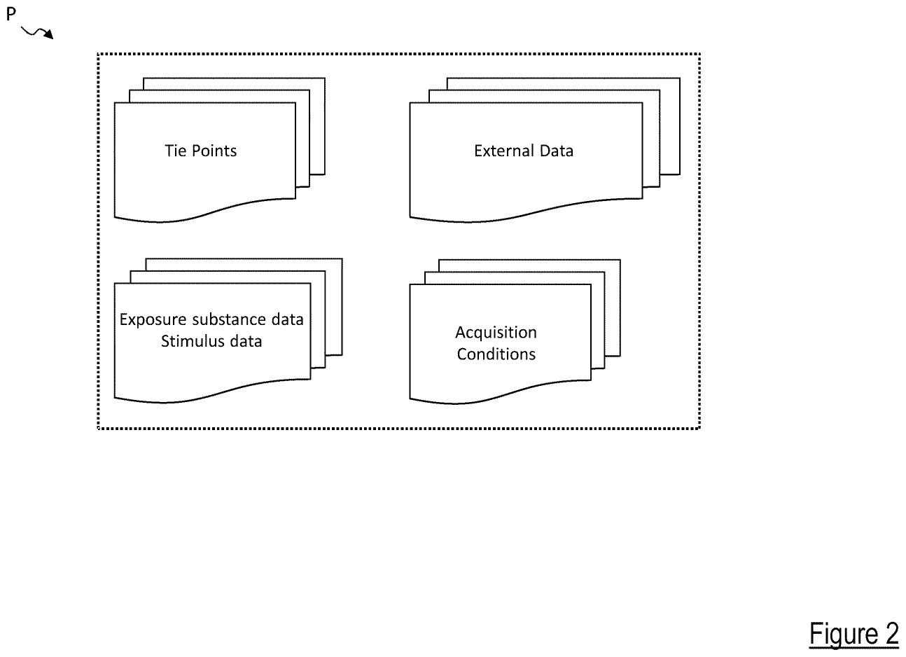

FIG. 2 shows a simplified structure of a survey protocol P according to embodiments of the present invention.

According to an embodiment, the survey protocol P comprises an indication of the tie points where the basal bioelectric signals and the corresponding induced bioelectric signals are to be detected.

According to an embodiment, as mentioned in the foregoing, the tie points may correspond to one or more body zones (each body zone comprising, for example, one or more organs and/or one or more glands and/or one or more parts of the body).

According to an embodiment, the tie points may correspond to one or more points of the body surface located along the meridians of the Chinese traditional medicine. Just as an example, the bioelectric signals detected in correspondence of one or more points of the body surface located along the meridians of the kidney, of the bladder or of the lung may provide (or contribute to provide) useful information about the kidney, the bladder or the lung, respectively.

According to an embodiment, the exact position of the tie point may be determined as the position in which the corresponding bioelectric signal has maximum intensity (indeed, the intensity of the bioelectric signal decreases around the tie point, approximately in a radius of 1-2 mm around it).

According to an embodiment, the survey protocol P comprises an indication of the exposure substance data (i.e., an indication of the exposure substance(s) to which the subject has to be exposed and/or exposure modes, such as exposure with contact or without contact and/or exposure times), and, possibly, of the stimulus data (i.e., an indication of the external stimuli to which the subject has to be exposed and of their application modes, such as order or sequence of application of the stimuli and/or procedure of exposure of the subject to the stimuli and/or intensity of application of the stimuli), an indication of the external data to be acquired, for example the nutritional and/or sports medicine data, the statistical data associated with one or more pathologies, the diagnostic/clinical data (such as ultrasound signals, and/or electrocardiographic signals, and/or electroencephalographic signals, and/or blood values, and/or urinary values, and/or stool values) and/or the measurements of the vital parameters (such as blood pressure and/or body temperature) and/or the healthcare data.

According to an embodiment, the survey protocol P comprises an indication of the acquisition conditions. Examples of acquisition conditions comprise, but are not limited to:
limitations on the intake of certain foods (for example, exciting drinks such as coffee and/or alcohol) for a predefined period of time before the survey;
limitations on the performance of certain activities (for example, intense sporting activity) for a predefined period of time before the survey (with the exception of surveys to be conducted in conditions of physical effort of the subject);
absence of emotional stress in the subject for a predefined period of time before the survey;
during the survey, absence of emotions in the subject (unless the survey contemplates it);
during the survey, control of the skin humidity of the subject;
intake of a predefined amount of water (for example 0.5-1 litre) in a predefined time interval before the survey;
during the survey, shielding of the subject and of the user of the system from electromagnetic disturbances potentially affecting the bioelectric signals and/or their acquisition.

Naturally, in order to satisfy local and specific requirements, a person skilled in the art may apply to the disclosure described above many logical and/or physical modifications and alterations. More specifically, although the present disclosure has been described with a certain degree of particularity with reference to preferred embodiments thereof, it should be understood that various omissions, substitutions and changes in the form and details as well as other embodiments are possible. In particular, different embodiments of the disclosure may even be practiced without the specific details set forth in the preceding description for providing a more thorough understanding thereof, on the contrary, well-known features may have been omitted or simplified in order not to encumber the description with unnecessary details. Moreover, it is expressly intended that specific elements and/or method steps described in connection with any disclosed embodiment of the disclosure may be incorporated in any other embodiment.

Particularly, similar considerations apply if the system has a different structure, comprises equivalent components. In any case, every component thereof may be separated into more elements, or two or more components may be combined together into a single element; moreover, each component may be replicated to support the execution of the corresponding operations in parallel. Moreover, unless specified otherwise, any interaction between different components generally does not need to be continuous, and it may be either direct or indirect through one or more intermediaries.

1. System for submitting a subject to a survey aimed at assessing a health state and/or a performance state of the subject, the system comprising:
one or more biosensors adapted to detect, in one or more points of the body surface, a plurality of bioelectric signals emitted by the body of the subject and associated with at least one body zone dependent on a purpose of the survey;
an acquisition module configured to acquire said plurality of bioelectric signals;
one or more substances to which the subject has to be exposed during the execution of the survey, wherein the one or more substances are configured for inducing, by exposure of the subject thereto, reactions in one or more molecules that are involved in one or more biochemical reactions that constitute a metabolic chain in the at least one body zone;
an analysis apparatus configured to implement one or more mathematical algorithms to:
determine a metabolic condition of the subject according to a difference between first bioelectric signals, among said plurality of bioelectric signals, spontaneously emitted by the body of the subject in the absence of exposure of the subject to said one or more substances, and second bioelectric signals, among said plurality of bioelectric signals, emitted by the body of the subject in response to the exposure of the subject to said one or more substances, wherein the exposure of the subject to said one or more substances comprises the placement of the one or more substances externally to the body of the subject and in proximity or contact with the body of the subject, wherein in presence of an altered metabolic condition the second bioelectric signals are different from the first bioelectric signals, as a result of said inducing reactions in said one or more molecules;
identify a correlation between the metabolic condition of the subject and at least one among:
nutritional data and/or sport medicine data;
statistical data associated with one or more pathologies;
diagnostic/clinical data of the subject obtained through one or more medical devices;
measurements of vital signs of the subject performed by one or more measurement modules;
healthcare data of the subject provided by one or more personal devices of the subject, and
anamnestic data of the subject, and
provide an outcome of the survey based on said metabolic condition and said correlation.

2. System according to claim 1, wherein the analysis apparatus comprises an artificial intelligence engine configured to operate based on a plurality of training data relating to a population of subjects, the analysis apparatus being configured to determine said metabolic condition and/or identify said correlation based on a similarity of the subject to other subjects in the population of subjects.

3. System according to claim 1, wherein the analysis apparatus is further configured to identify possible undesired electrical contributions provided to said plurality of bioelectric signals by one or more body zones of the subject different from said at least one body zone, and compensate said plurality of bioelectric signals according to said undesired electrical contributions, the analysis apparatus being configured to determine said metabolic condition of the subject and/or identify said correlation according to said plurality of compensated electrical signals.

4. System according to claim 1, wherein the analysis apparatus is further configured to determine a validity and/or quality of the plurality of bioelectric signals.

5. System according to claim 1, wherein the analysis apparatus is further configured to perform a pre-processing of the plurality of bioelectric signals for determining at least one bioelectric emission parameter, the analysis apparatus being configured to determine said metabolic condition of the subject and/or identify said correlation according to said at least one bioelectric emission parameter.

6. System according to claim 5, wherein said at least one bioelectric emission parameter comprises one or more among:
an average bioelectric emission index;
a total bioelectric emission index;
statistical variance and/or standard deviation of the plurality of bioelectric signals;
relative differences between bioelectric emissions associated with different body zones of said at least one body zone of the subject;
a response index indicative of an impact of at least one of said one or more external stimuli on at least one of the at least one body zone;
a relative comparison between sub-sets of the acquired bioelectric signals.

7. System according to claim 1, comprising one or more external stimuli sources each one adapted to generate one or more external stimuli to which the subject has to be exposed during the execution of the survey, the second bioelectric signals being emitted by the body of the subject further in response to the exposure of the subject to at least one of said one or more external stimuli.

8. System according to claim 7, wherein said external stimuli sources comprise at least one among:
one or more acoustic stimulus generators;
one or more luminous and/or/visual stimulus generators, and
one or more olfactory stimulus generators.

9. System according to claim 1, wherein each biosensor comprises or incorporates said one or more substances.

10. System according to claim 1, further comprising said one or more medical devices and/or said one or more measurement modules.

11. System according to claim 1, wherein said medical devices comprise one or more among:
an ultrasound machine;
an electrocardiograph;
an electroencephalograph;
a blood analysis apparatus;
a urinalysis device;
a stool analysis device.
